(19) United States
(12) Patent Application Publication
(10) Pub. No.: US 2017/0135655 A1
(43) Pub. Date: May 18, 2017

(54) FACIAL TEXTURE MAPPING TO VOLUME IMAGE

(71) Applicant: Carestream Health, Inc., Rochester, NY (US)

(72) Inventors: Wei Wang, Shanghai (CN); Zhaohua Liu, Shanghai (CN); Guijian Wang, Shanghai (CN); Jean-Marc Inglese, Bussy-Saint-Georges (FR)

(21) Appl. No.: 15/319,762

(22) PCT Filed: Aug. 8, 2014
(86) PCT No.: PCT/CN2014/083989

§ 371 Date: Dec. 17, 2016

(51) Int. Cl. A61B 6/14; G06T 15/04; G06K 9/46; A61C 9/00; A61B 1/24; A61B 6/03; A61B 6/00; A61C 7/00; G06T 17/20; G06T 7/00

(52) U.S. Cl. CPC A61B 6/14; G06T 17/20; G06T 15/04; G06K 9/4604; G06T 7/0012; A61B 1/24; A61B 6/032; A61B 6/4085; A61C 7/002; A61C 9/0053; A61B 6/5247; G06T 2207/10028; G06T 2207/10124; G06T 2207/10081

(57) ABSTRACT

A method for forming a 3-D facial model obtains a reconstructed radiographic image volume of a patient and extracts a soft tissue surface of the patient's face from the image volume and forms a dense point cloud of the extracted surface. Reflection images of the face are acquired using a camera, wherein each reflection image has a different corresponding camera angle with respect to the patient. Calibration data is calculated for one or more of the reflection images. A sparse point cloud corresponding to the reflection images is formed by processing the reflection images using multi-view geometry. The sparse point cloud is registered to the dense point cloud and a transformation calculated between reflection image texture data and the dense point cloud. The calculated transformation is applied for mapping texture data from the reflection images to the dense point cloud to form a texture-mapped volume image that is displayed.

### FIELD OF THE INVENTION

The invention relates generally to 3-dimensional (3-D) imaging and more particularly relates to methods incorporating textural information to a 3-D representation of the human face to form a 3-D facial model.

### BACKGROUND OF THE INVENTION

Orthodontic procedures and orthognathic surgery seek to correct dentofacial conditions including structural asymmetry, aesthetic shortcomings, and alignment and other functional problems that relate to the shape of the patient's face and jaws. One tool that can be of particular value for practitioners skilled in orthodontics and related fields is photorealistic modeling. Given a facial model displayed as an accurate volume rendition of the patient's head, showing the structure as well as the overall surface appearance or texture of the patient's face, the practitioner can more effectively visualize and plan a treatment procedure that provides both effective and pleasing results.

Generating a volume image that provides a suitable visualization of the human face for corrective procedures relating to teeth, jaws, and related dentition uses two different types of imaging. A volume image that shows the shape and dimensions of the head and jaws structure is obtained using computed tomography (CT), such as cone-beam computed tomography (CBCT), or other volume imaging method, including magnetic resonance imaging (MRI) or magnetic resonance tomography (MRT). The volume image, however, has no color or perceptible textural content and would not, by itself, be of much value for showing simulated results to a patient or other non-practitioner, for example. To provide useful visualization that incorporates the outer, textural surface of the human face, a camera is used to obtain reflectance or “white light” images. The color and texture information from the camera images is then correlated with volume image information in order to provide an accurate rendition usable by the orthodontics practitioner.

Solutions that have been proposed for addressing this problem include methods that provide at least some level of color and texture information that can be correlated with volume image data from CBCT or other scanned image sources. These conventional solutions include so-called range-scanning methods.

Reference is made to U.S. Patent Application Publication No. 2012/0300895 entitled “DENTAL IMAGING APPARATUS” by Koivisto et al. that combines texture information from reflectance images along with surface contour data from a laser scan.

Reference is made to U.S. Patent Application Publication No. 2013/0163718 entitled “DENTAL X-RAY DEVICE WITH IMAGING UNIT FOR SURFACE DETECTION AND METHOD FOR GENERATING A RADIOGRAPH OF A PATIENT” by Lindenberg et al. that describes using a masking edge for scanning to obtain contour and color texture information for combination with x-ray data.

The '0895 Koivisto et al. and '3718 Lindberg et al. patent applications describe systems that can merge volume image data from CBCT or other scanned image sources with 3-D surface data that is obtained from 3-D range-scanning devices. The range scanning devices can provide some amount of contour data as well as color texture information. However, the solutions that are described in these references can be relatively complex and costly. Requirements for additional hardware or other specialized equipment with this type of approach add cost and complexity and are not desirable for the practitioner.

A dental imaging system from Dolphin Imaging Software (Chatsworth, Calif.) provides features such as a 2-D facial wrap for forming a texture map on the facial surface of a 3-D image from a CBCT, CT or MRI scan.

Reference is made to a paper by Iwakiri, Yorioka, and Kaneko entitled “Fast Texture Mapping of Photographs on a 3D Facial Model” in Image and Vision Computing NZ, November 2003, pp. 390-395.

Both the Dolphin software and the Iwakiri et al. method map 2-D image content to 3-D CBCT volume image data. While such systems may have achieved certain degrees of success in particular applications, there is room for improvement. For example, the Dolphin software user, working with a mouse, touch screen, or other pointing device, must accurately align and re-position the 2-D content with respect to 3-D content that appears on the display screen. Furthermore, imprecise registration of 2-D data that provides information on image texture to the 3-D volume data compromises the appearance of the combined data.

Thus, there is a need for apparatus and method for accurately generating a volume image that provides accurate representation of textural features.

### SUMMARY OF THE INVENTION

An object of the present disclosure is to advance the art of volume imaging, particular for orthodontic patients.

Another object of the present disclosure is to provide a system that does not require elaborate, specialized hardware for providing a 3-D model of a patient's head. Advantageously, methods disclosed herein can be executed using existing CBCT hardware, providing accurate mapping of facial texture information to volume 3-D data.

These objects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

According to one aspect of the invention, there is provided a method for forming a 3-D facial model, the method executed at least in part on a computer and comprising:
obtaining a reconstructed radiographic image volume of at least a portion of the head of a patient;
extracting a soft tissue surface of the patient's face from the reconstructed radiographic image volume and forming a dense point cloud corresponding to the extracted soft tissue surface;
acquiring a plurality of reflection images of the face using a camera, wherein each reflection image has a different corresponding camera angle with respect to the patient and calculating calibration data for the camera for one or more of the reflection images;
forming a sparse point cloud corresponding to the reflection images by processing the reflection images using multi-view geometry and the calculated calibration data;
registering the sparse point cloud to the dense point cloud and calculating a transformation between reflection image texture data and the dense point cloud;
applying the calculated transformation for mapping texture data from the plurality of reflection images to the dense point cloud to form a texture-mapped volume image;
and
displaying the texture-mapped volume image.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is a logic flow diagram that shows a processing sequence for texture mapping to provide a volume image of a patient's face using 2-D to 3-D image registration.

FIG. 2 is a schematic diagram that shows portions of a volume image.

FIGS. 3A and 3B show feature points from 3-D volume data that can be used to generate a depth map of the patient's face.

FIGS. 4A and 4B show calculation of feature points from 2-D reflectance image data.

FIG. 5 is a schematic diagram that shows principles of 2-D to 3-D image registration according to methods that use 2-D to 3-D image registration.

FIG. 6 is a schematic diagram that shows forming a texture-mapped volume image according methods that use 2-D to 3-D image registration.

FIG. 7 is a logic flow diagram that shows steps in a texture mapping process according to an embodiment of the present invention.

FIG. 8 is a schematic diagram that shows generation of reflectance image data used for a sparse 3-D model.

FIG. 9 is a schematic diagram that shows generation of a sparse 3-D model according to a number of reflectance images.

FIG. 10 is a schematic diagram that shows matching the 3-D data from reflective and radiographic sources.

FIG. 11 is a schematic diagram that shows an imaging apparatus for obtaining a 3-D facial model from volume and reflectance images according to an embodiment of the present disclosure.

### DETAILED DESCRIPTION OF EXEMPLARY EMBODIMENTS

The following is a detailed description of exemplary embodiments of the application, reference being made to the drawings in which the same reference numerals identify the same elements of structure in each of the several figures.

In the drawings and text that follow, like components are designated with like reference numerals, and similar descriptions concerning components and arrangement or interaction of components already described are omitted. Where they are used, the terms “first”, “second”, and so on, do not necessarily denote any ordinal or priority relation, but are simply used to more clearly distinguish one element from another.

In the context of the present disclosure, the term “volume image” is synonymous with the terms “3-dimensional image” or “3-D image”. 3-D volume images can be cone-beam computed tomography (CBCT) as well as fan-beam CT images, as well as images from other volume imaging modalities, such as magnetic resonance imaging (MM).

For the image processing steps described herein, the terms “pixels” for picture image data elements, conventionally used with respect 2-D imaging and image display, and “voxels” for volume image data elements, often used with respect to 3-D imaging, can be used interchangeably. It should be noted that the 3-D volume image is itself synthesized from image data obtained as pixels on a 2-D sensor array and displays as a 2-D image from some angle of view. Thus, 2-D image processing and image analysis techniques can be applied to the 3-D volume image data. In the description that follows, techniques described as operating upon pixels may alternately be described as operating upon the 3-D voxel data that is stored and represented in the form of 2-D pixel data for display. In the same way, techniques that operate upon voxel data can also be described as operating upon pixels.

In the context of the present disclosure, the noun “projection” may be used to mean “projection image”, referring to the 2-D radiographic image that is captured and used to reconstruct the CBCT volume image, for example.

The term “set”, as used herein, refers to a non-empty set, as the concept of a collection of elements or members of a set is widely understood in elementary mathematics. The term “subset”, unless otherwise explicitly stated, is used herein to refer to a non-empty proper subset, that is, to a subset of the larger set, having one or more members. For a set S, a subset may comprise the complete set S. A “proper subset” of set S, however, is strictly contained in set S and excludes at least one member of set S.

As used herein, the term “energizable” relates to a device or set of components that perform an indicated function upon receiving power and, optionally, upon receiving an enabling signal.

The term “reflectance image” refers to an image or to the corresponding image data that is captured by a camera using reflectance of light, typically visible light. Image texture includes information from the image content on the distribution of color, shadow, surface features, intensities, or other visible image features that relate to a surface, such as facial skin, for example.

Cone-beam computed tomography (CBCT) or cone-beam CT technology offers considerable promise as one type of tool for providing diagnostic quality 3-D volume images. Cone-beam X-ray scanners are used to produce 3-D images of medical and dental patients for the purposes of diagnosis, treatment planning, computer aided surgery, etc. Cone-beam CT systems capture volume data sets by using a high frame rate flat panel digital radiography (DR) detector and an x-ray source, typically both affixed to a gantry or other transport, that revolve about the subject to be imaged. The CT system directs, from various points along its orbit around the subject, a divergent cone beam of x-rays through the subject and to the detector. The CBCT system captures projection images throughout the source-detector orbit, for example, with one 2-D projection image at every degree increment of rotation. The projections are then reconstructed into a 3-D volume image using various techniques. Among the most common methods for reconstructing the 3-D volume image from 2-D projections are filtered back projection (FBP) and Feldkamp-Davis-Kress (FDK) approaches.

Embodiments of the present disclosure use a multi-view imaging technique that obtains 3-D structural information from 2-D images of a subject, taken at different angles about the subject. Processing for multi-view imaging can employ “structure-from-motion” (SFM) imaging technique, a range imaging method that is familiar to those skilled in the image processing arts. Multi-view imaging and some applicable structure-from-motion techniques are described, for example, in U.S. Patent Application Publication No. 2012/0242794 entitled “Producing 3D images from captured 2D video” by Park et al., incorporated herein in its entirety by reference.

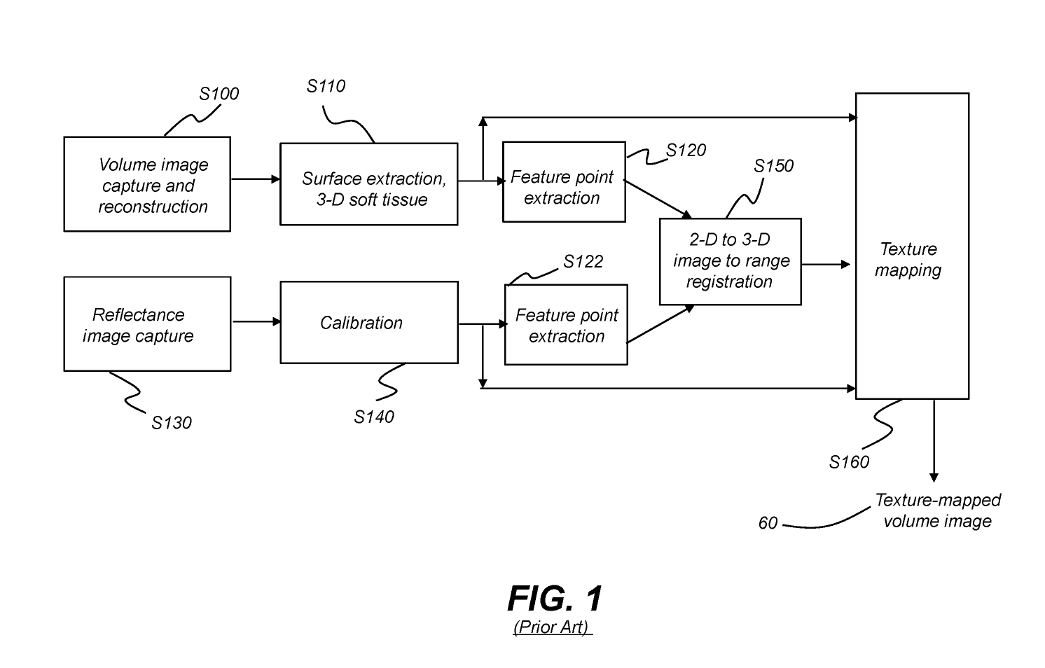

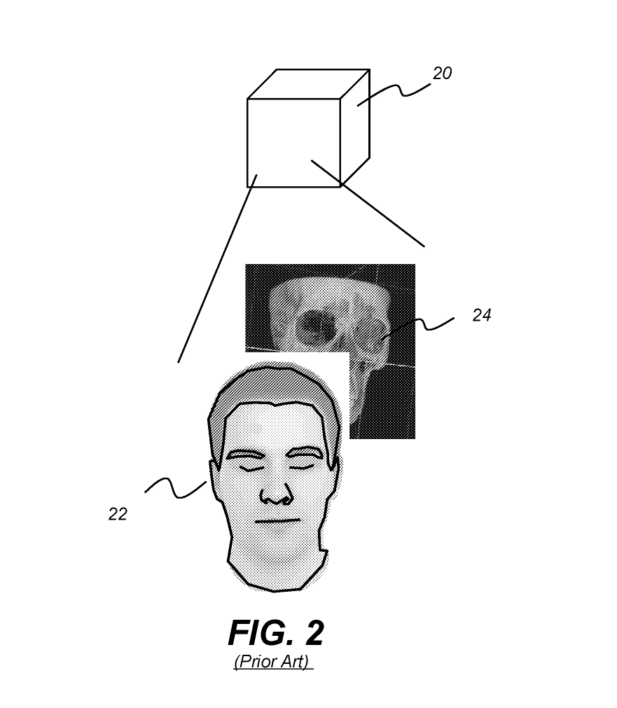

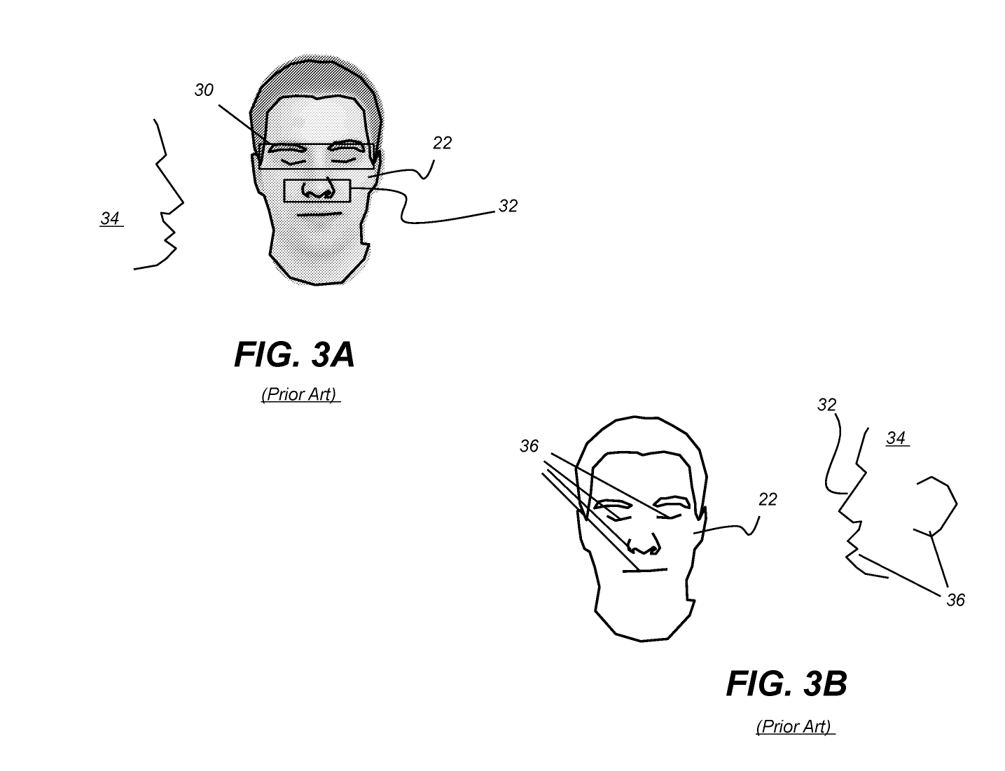

The logic flow diagram of FIG. 1 shows a conventional processing sequence for texture mapping to provide a volume image of a patient's face using 2-D to 3-D image registration. Two types of images are initially obtained. A volume image capture and reconstruction step S100 acquires a plurality of 2-D radiographic projection images and performs 3-D volume reconstruction, as described. A surface extraction step S110 extracts surface shape, position, and dimensional data for soft tissue that lies on the outer portions of the reconstructed volume image. As shown in FIG. 2, a volume image 20 can be segmented into an outer soft tissue surface 22 and a hard tissue structure 24 that includes skeletal and other dense features; this segmentation can be applied using techniques familiar to those skilled in the imaging arts. A feature point extraction step S120 then identifies feature points of the patient from the extracted soft tissue. As shown in FIGS. 3A and 3B, feature points 36 from the volume image can include eyes 30, nose 32, and other prominent edge and facial features. Detection of features and related spatial information can help to provide a depth map 34 of the face soft tissue surface 22.

Continuing with the FIG. 1 sequence, multiple reflectance images of the patient are captured in a reflectance image capture step S130. Each reflectance image has a corresponding camera angle with respect to the patient; each image is acquired at a different camera angle. A calibration step S140 calculates the intrinsic parameters of a camera model, so that a standardized camera model can be applied for more accurately determining position and focus data. In the context of procedures described in the present disclosure, calibration relates to camera resectioning, rather than just to color or other photometric adjustment. The resectioning process estimates camera imaging characteristics according to a model of a pinhole camera, and provides values for a camera matrix. This matrix is used to correlate real-world 3-D spatial coordinates with camera 2-D pixel coordinates. Camera resectioning techniques are familiar to those skilled in the computer visualization arts.

The reflectance image and calibration data in the FIG. 1 sequence are then input to a feature point extraction step S122 that identifies feature points of the patient from the reflectance image.

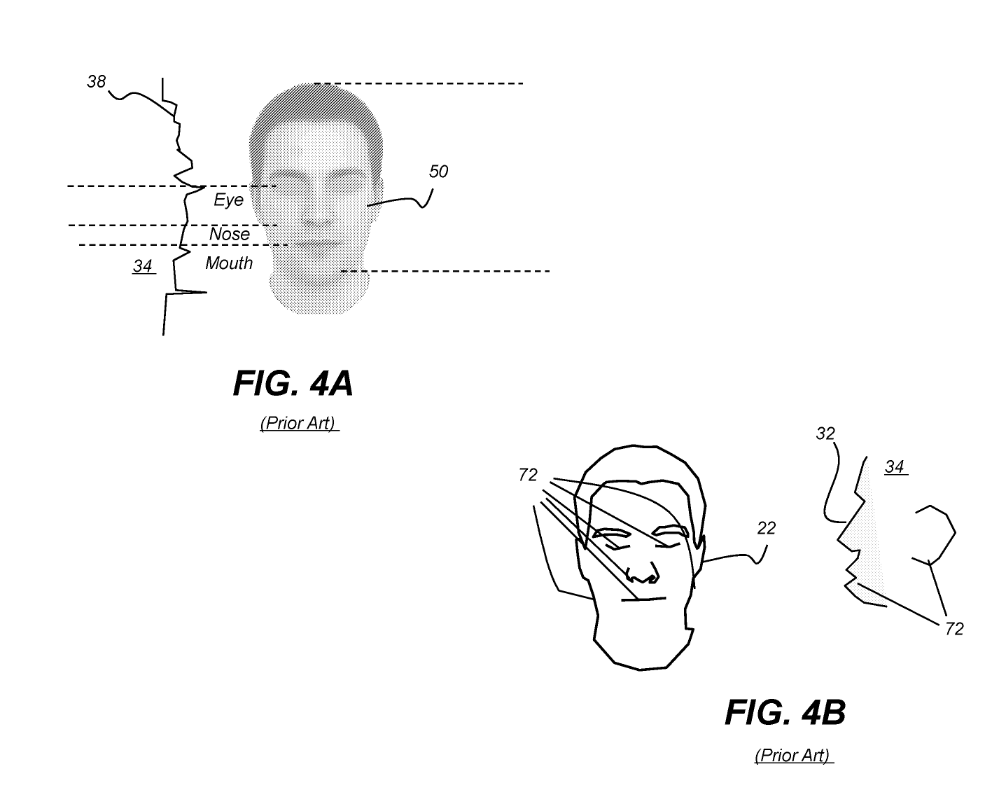

FIGS. 4A and 4B show feature points 72 extraction from the reflectance image. A horizontally projected sum 38 for feature point detection relative to a row of pixels is shown; a vertically projected sum for pixel columns can alternately be provided for this purpose. Various edge operators, such as familiar Sobel filters, can be used to assist in automatic edge detection.

Identifying feature points 36 and 72 helps to provide the needed registration between 2-D and 3-D image data in a subsequent registration step S150 of the FIG. 1 sequence. Registration step S150 then maps the detected feature points 72 from the 2-D reflectance image content to detected feature points 36 from the 3-D range image data.

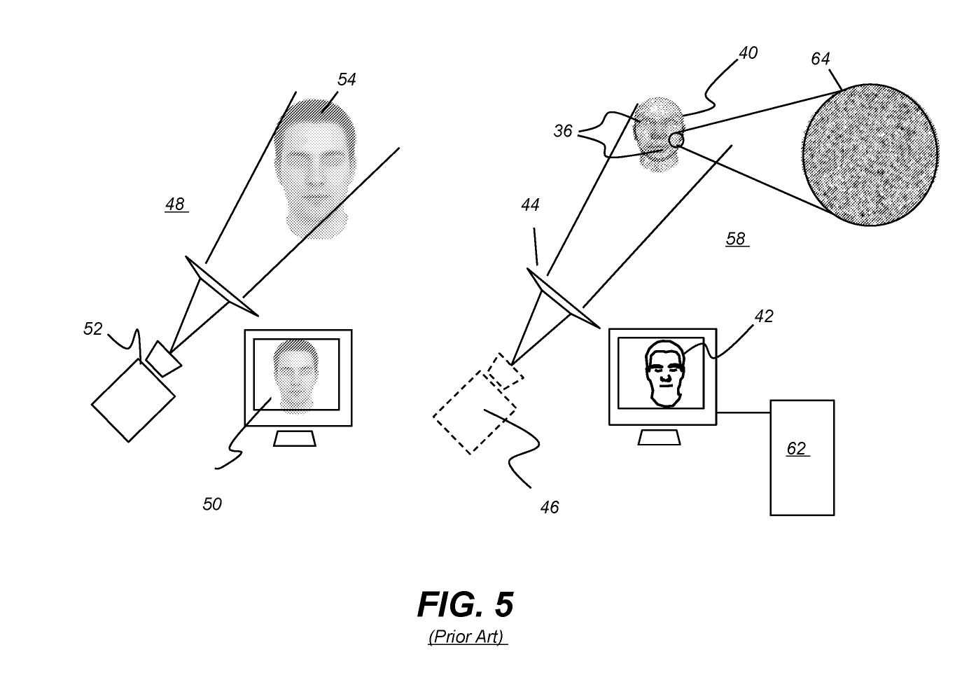

FIG. 5 shows this registration process in schematic form. A polygon model 40 is generated from 3-D volume data of soft tissue surface 22. Using the arrangement of FIG. 5, an imaging apparatus 48 uses camera 52 to obtain reflectance (white light) images 50 of a patient 54. A virtual system 58 uses a computer 62 to apply the registration parameter calculation in step S150 and texture mapping step S160, mapping texture content to a polygon model 40 generated from 3-D volume image that has been previously generated by computer 62 logic.

FIG. 5 shows an enlarged portion of the patient's face with polygons 64. Reflectance image 50 captured by camera 52 is mapped to a projected image 42 that has been generated from the polygon model 40 using feature points 36 as described previously with reference to FIGS. 3A-4B. Projected image 42 is calculated from polygon model 40 by projection onto a projection plane 44, modeled as the image plane of a virtual camera 46, shown in dashed outline. For alignment of the reflectance and virtual imaging systems shown in FIG. 5, feature points 36 and 72, such as eyes, mouth, edges, and other facial structures can be used.

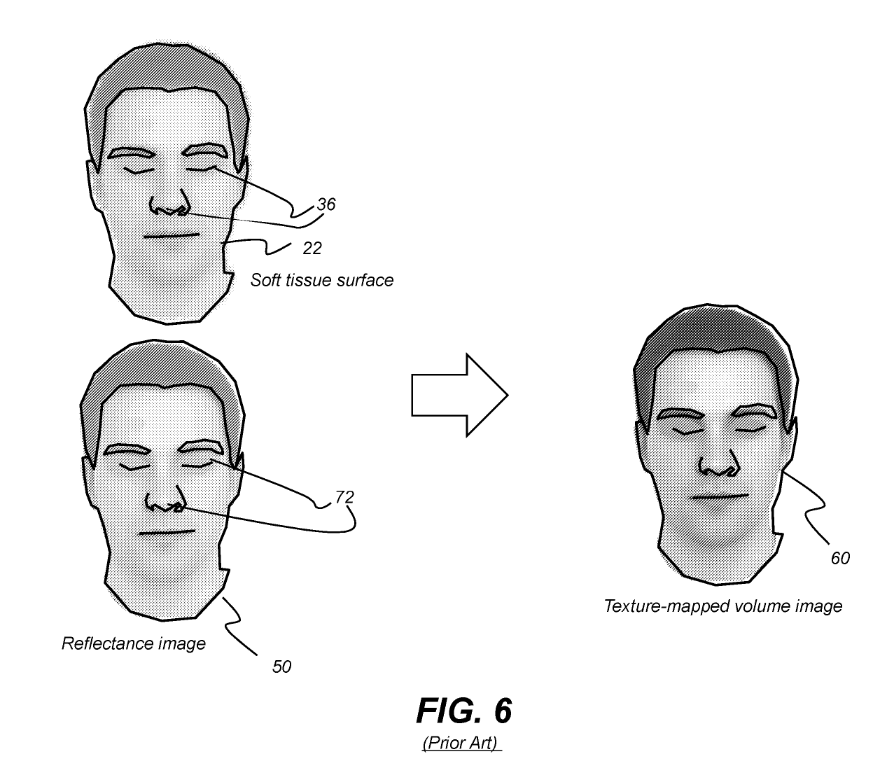

At the conclusion of the FIG. 1 sequence, a texture mapping step S160 generates a texture-mapped volume image 60 from soft tissue surface 22 and reflectance image 50 as shown in FIGS. 5 and 6. Texture mapping step S160 uses the surface extraction and camera calibration data for soft tissue surface 22 and reflectance image 50 and uses this data to combine the soft tissue surface 22 and reflectance image 50 using registration step S150 results. The generated output, texture-mapped volume image 60 can then be viewed from an appropriate angle and used to assist treatment planning.

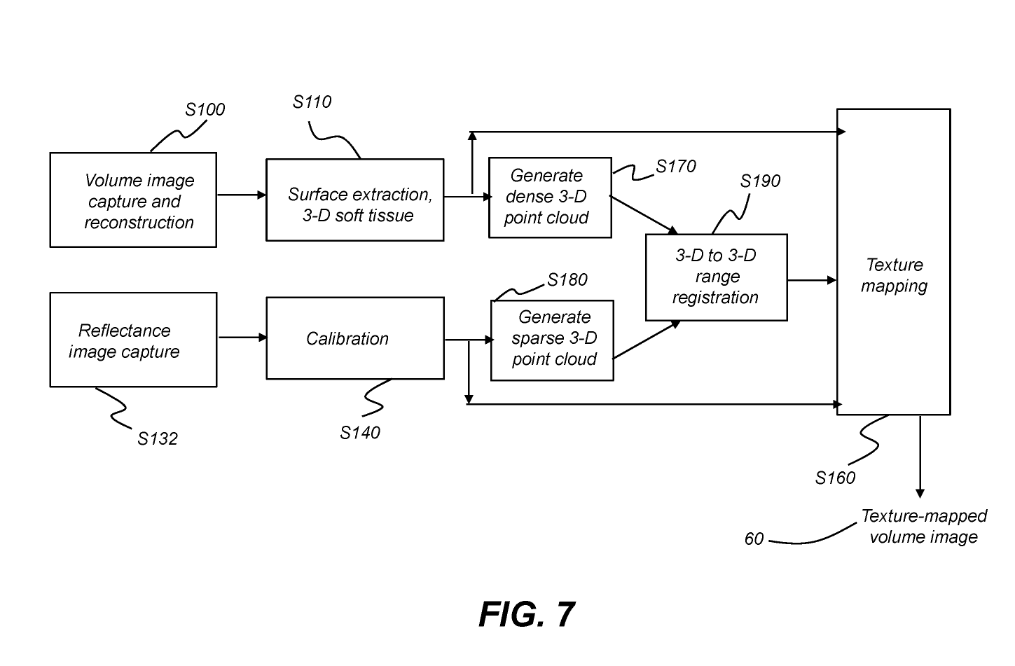

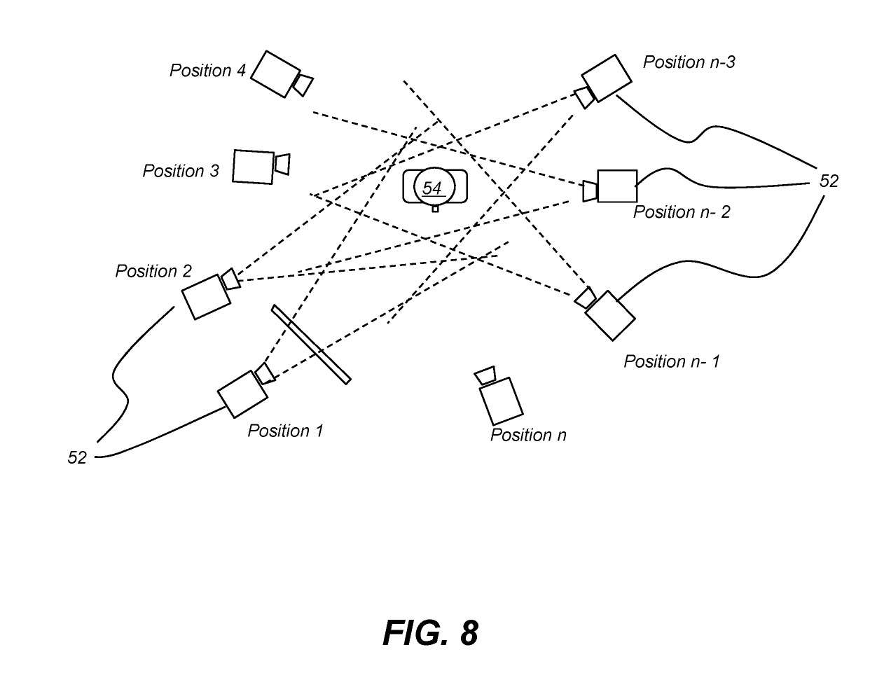

The logic flow diagram of FIG. 7 shows a sequence for generating a texture-mapped volume image 60 using techniques of multi-view geometry according to an exemplary embodiment of the present disclosure. A number of the initial steps are functionally similar to those described with respect to FIGS. 1-6. Volume image capture and reconstruction step S100 acquires a plurality of 2-D radiographic projection images and performs 3-D volume reconstruction, as described previously. Surface extraction step S110 extracts surface shape, position, and dimensional data for soft tissue that lies on the outer portions of the reconstructed volume image. Step S110 generates soft tissue surface 22 and underlying hard tissue structure 24 that includes skeletal and other dense features (FIG. 2). Multiple reflectance images of the patient are captured in a reflectance image capture step S132. As shown in FIG. 8, each reflectance image that is acquired has a corresponding camera angle with respect to the patient; each image is acquired at a different camera angle. In FIG. 8, camera angles correspond to positions 1, 2, 3, 4, . . . n, n−3, etc. Calibration step S140 of FIG. 7 calculates the intrinsic parameters of a camera model, so that a standardized camera model can be applied for more accurately determining position and focus data. Calibration can relate to camera resectioning, rather than to color or other photometric adjustment. The resectioning process estimates camera imaging characteristics according to a model of a pinhole camera, and provides values for a camera matrix. This matrix is primarily geometric, used to correlate real-world 3-D spatial coordinates with camera 2-D pixel coordinates.

Continuing with the sequence of FIG. 7, the method executes an exemplary dense point cloud generation step S170 in order to generate points in space that correspond to the 3-D soft tissue surface of the patient. This generates a dense 3-D model in the form of a dense point cloud; the terms “3-D model” and “point cloud” are used synonymously in the context of the present disclosure. The dense point cloud is formed using techniques familiar to those skilled in the volume imaging arts for forming a Euclidean point cloud and relates generally to methods that identify points corresponding to voxels on a surface. The dense point cloud is thus generated using the reconstructed volume data, such as CBCT data. Surface points from the reconstructed CBCT volume are used to form the dense point cloud for this processing. The dense point cloud information serves as the basis for a polygon model at high density for the head surface.

The reflectance images then provide a second point cloud for the face surface of the patient. In an exemplary sparse point cloud generation step S180, the reflectance images obtained in reflectance image capture step S132 are used to generate another point cloud, termed a sparse point cloud, with relatively fewer surface points defined when compared to the dense point cloud for the same surface. In the context of the present disclosure, for a given surface such as a face, a sparse point cloud for that surface has fewer point spatial locations than does a dense point cloud that was obtained from a volume image. Typically, though not necessarily, the dense point cloud has significantly more points than does the sparse point cloud. Both point clouds are spatially defined and constrained by the overall volume and shape associated with the facial surface of the patient. The actual point cloud density for the dense point cloud depends, at least in part, on the overall resolution of the 3-D volume image. Thus, for example, where the isotropic resolution for a volume image is 0.5 mm, the corresponding resolution of the dense point cloud is constrained so that points in the dense point cloud are no closer than 0.5 mm apart. In typical practice, the point cloud that is generated for the same subject from a succession of 2-D images using structure-from-motion or related multi-view geometry techniques is sparse by comparison with the point cloud generated using volume imaging.

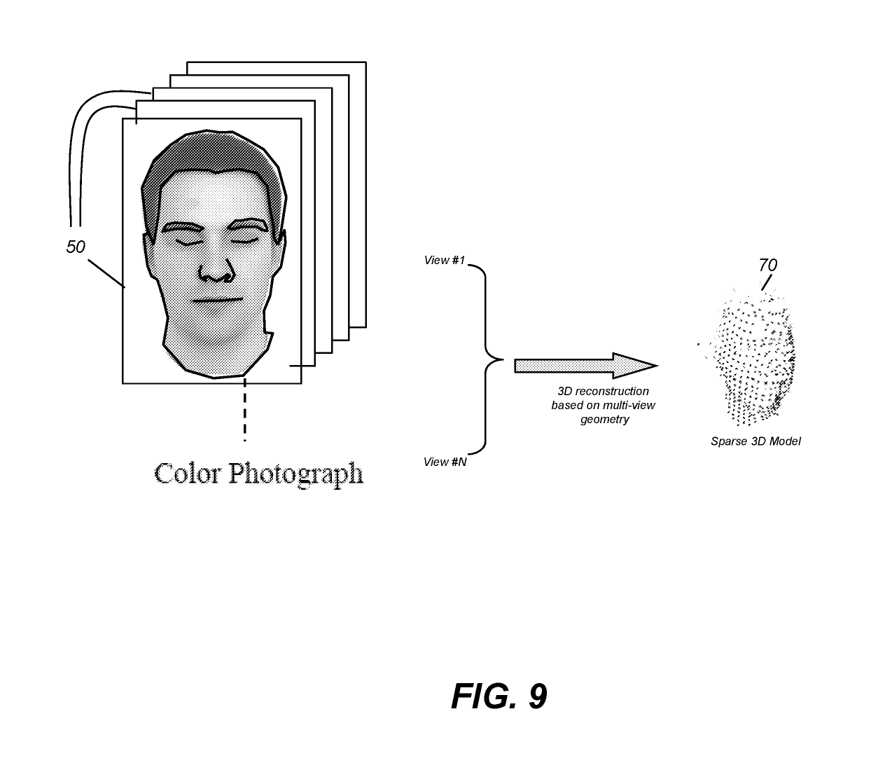

To generate the sparse point cloud, the system applies multi-view geometry methods to the reflectance images 50 acquired in step S132. Step 180 processing is shown in FIG. 9, using reflectance images 50 for obtaining sparse point cloud data. A sparse 3-D model 70 is generated from the reflectance images 50. Sparse 3-D model 70 can optionally be stored in a memory. Forming the sparse cloud can employ structure-from-motion (SFM) methods, for example.

Structure from motion (SFM) is a range imaging technique known to those skilled in the image processing arts, particularly with respect to computer vision and visual perception. SFM relates to the process of estimating three-dimensional structures from two-dimensional image sequences which may be coupled with local motion signals. In biological vision theory, SFM has been related to the phenomenon by which the human viewer can perceive and reconstruct depth and 3-D structure from the projected 2-D (retinal) motion field of a moving object or scene. According to an embodiment of the present invention, the sparse point cloud 70 can be recovered from a number of reflectance images 50 obtained in step S132 (FIG. 7) and from camera calibration data. Sparse point cloud generation step S180 represents the process for sparse 3-D model 70 generation.

References to Structure-from-motion (SFM) image processing techniques include U.S. Patent Application Publication No. 2013/0265387 A1 entitled “Opt-Keyframe Reconstruction for Robust Video-Based Structure from Motion” by Hailin Jin.

References to 2-D to 3-D image alignment include U.S. Patent Application Publication No. 2008/0310757 entitled “System and Related Methods for Automatically Aligning 2D Images of a Scene to a 3D Model of the Scene” to Wolberg et al.

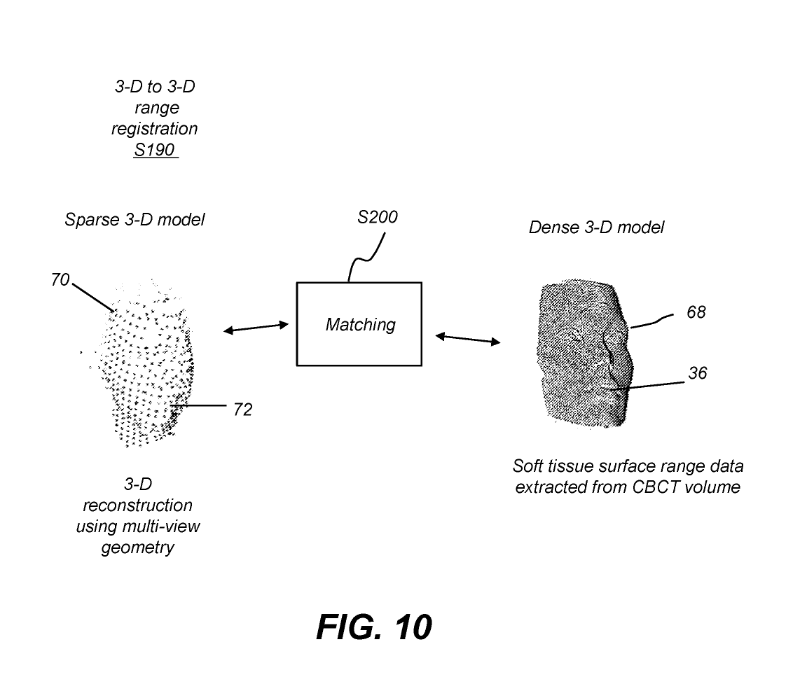

As shown in FIG. 7, a registration step S190 provides 3-D to 3-D range registration between the sparse and dense point clouds. FIG. 10 shows a matching function S200 of registration step S190 that matches the sparse 3-D model 70 with its corresponding dense 3-D model 68. Matching function S200 uses techniques such as view angle computation between features 72 and 36 and polygon approximations, alignment of centers of gravity or mass, and successive operations of coarse and fine alignment matching to register and adjust for angular differences between dense and sparse point clouds. Registration operations for spatially correlating the dense and sparse point clouds 68 and 70 include rotation, scaling, translation, and similar spatial operations that are familiar to those skilled in the imaging arts for use in 3-D image space. Once this registration is complete, texture mapping step S160 uses the point cloud structures that represent the head and facial surfaces and may use a polygon model that is formed using the point cloud registration data in order to generate texture-mapped volume image 60.

According to one embodiment of the present disclosure, texture mapping step S160 can proceed as follows:
(i) Calculate matching function S200 (FIG. 10) to achieve spatial correspondence between the dense 3-D point cloud of dense 3-D model 68 that is obtained from the volume image and the sparse 3-D point cloud of sparse 3-D model 70 that is generated from the reflectance images 50. Transform calculations using scaling, rotation, and translation can then be used to register or correlate a sufficient number of points from the sparse 3-D model 70 to dense 3-D model 68.
(ii) Calculate the correspondence between the reflectance images 50 obtained from different positions (FIG. 8) and the sparse 3-D model 70 (FIG. 9). Points in reflectance images 50 are mapped to the sparse 3-D model 70.
(iii) Based on the calculation results of steps (i) and (ii), calculate the correspondence between the reflectance image(s) 50 obtained from different positions (FIG. 8) and the dense 3-D point cloud of dense 3-D model 68 that is obtained from the volume image. One or more polygons can be formed using points that are identified in the volume image data as vertices, generating a polygon model of the skin surface. Transform calculations using scaling, rotation, and translation can then be used to correlate points and polygonal surface segments on the reflectance images 50 and the dense 3-D model 68.
(iv) The correspondence results of step (iii) provide the information that is needed to allow texture mapping step S160 to map reflection image 50 content to the volume image content, polygon by polygon, according to mappings of surface points.

Generation of a polygon model from a point cloud is known to those skilled in the imaging arts. One type of polygon model generation is described, for example, in U.S. Pat. No. 8,207,964 entitled “Methods and apparatus for generating three-dimensional image data models” to Meadow et al. More generally, polygons are generated by connecting nearest-neighbor points within the point cloud as vertices, forming contingent polygons of three or more sides that, taken together, define the skin surface of the patient's face. Polygon model generation provides interconnection of vertices, as described in U.S. Pat. No. 6,975,750 to Han et al., entitled “System and method for face recognition using synthesized training images.” Mapping of the texture information to the polygon model from the reflectance images forms the texture-mapped volume image.

In displaying the texture-mapped volume image, an optional measure of transparency can be provided for the texture components, to allow improved visibility of internal structures, such as jaws, teeth, and other dentition elements.

An embodiment of the present invention can be integrated into 3-D Visual Treatment Objective (VTO) software, used in orthognathic surgery, for example.

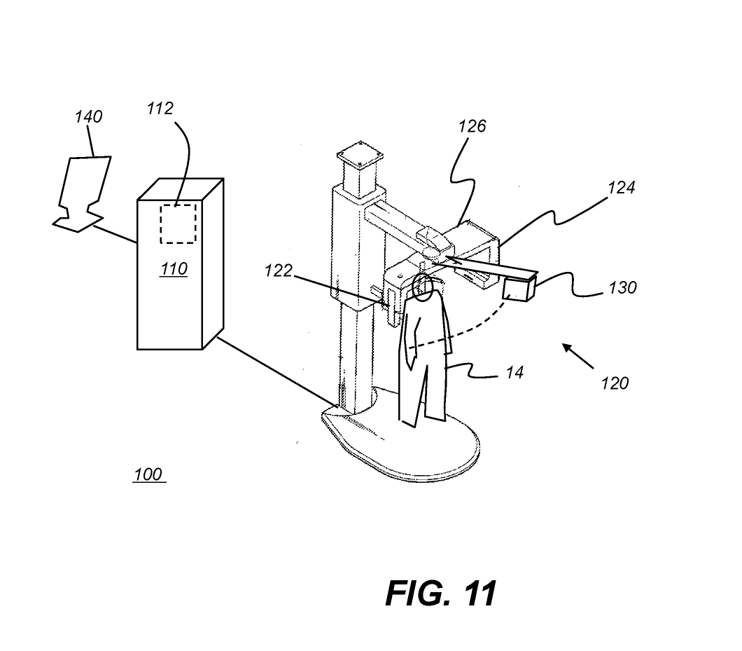

The schematic diagram of FIG. 11 shows an imaging apparatus 100 for obtaining a 3-D facial model from volume and reflectance images according to an embodiment of the present disclosure. A patient 14 is positioned within a CBCT imaging apparatus 120 that has a radiation source 122 and a detector 124 mounted on a rotatable transport 126 that acquires a series of radiographic images. Imaging apparatus 100 also has a camera 130, which may be integrated with the CBCT imaging apparatus 120 or may be separately mounted or even hand-held. Camera 130 acquires the reflectance or white-light images of patient 14 for use by the SFM or other multi-view imaging logic. A control logic processor 110 is in signal communication with imaging apparatus 120 for acquiring and processing both the CBCT and reflectance image content according to software that can form a processor 112 for executing multi-view imaging and performing at least the point cloud generation, registration, and matching functions described herein, along with mapping steps for generating and displaying the texture-mapped volume image on a display 140.

Consistent with one embodiment, the present invention utilizes a computer program with stored instructions that perform on image data accessed from an electronic memory. As can be appreciated by those skilled in the image processing arts, a computer program of an embodiment of the present invention can be utilized by a suitable, general-purpose computer system, such as a personal computer or workstation. However, many other types of computer systems can be used to execute the computer program of the present invention, including networked processors. The computer program for performing the method of the present invention may be stored in a computer readable storage medium. This medium may comprise, for example; magnetic storage media such as a magnetic disk such as a hard drive or removable device or magnetic tape; optical storage media such as an optical disc, optical tape, or machine readable bar code; solid state electronic storage devices such as random access memory (RAM), or read only memory (ROM); or any other physical device or medium employed to store a computer program. The computer program for performing the method of the present invention may also be stored on computer readable storage medium that is connected to the image processor by way of the internet or other communication medium. Those skilled in the art will readily recognize that the equivalent of such a computer program product may also be constructed in hardware.

It should be noted that the term “memory”, equivalent to “computer-accessible memory” in the context of the present disclosure, can refer to any type of temporary or more enduring data storage workspace used for storing and operating upon image data and accessible to a computer system. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Displaying an image requires memory storage. Display data, for example, is typically stored in a temporary storage buffer that is directly associated with a display device and is periodically refreshed as needed in order to provide displayed data. This temporary storage buffer can also be considered to be a memory, as the term is used in the present disclosure. Memory is also used as the data workspace for executing and storing intermediate and final results of calculations and other processing. Computer-accessible memory can be volatile, non-volatile, or a hybrid combination of volatile and non-volatile types.

It will be understood that the computer program product of the present invention may make use of various image manipulation algorithms and processes that are well known. It will be further understood that the computer program product embodiment of the present invention may embody algorithms and processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with the computer program product of the present invention, are not specifically shown or described herein and may be selected from such algorithms, systems, hardware, components and elements known in the art.

In one exemplary embodiment, a method for forming a 3-D facial model can be executed at least in part on a computer and can include obtaining a reconstructed computed tomography image volume of at least a portion of the head of a patient; extracting a soft tissue surface of the patient's face from the reconstructed computed tomography image volume and forming a dense point cloud corresponding to the extracted soft tissue surface; acquiring a plurality of reflection images of the face, wherein each reflection image in the plurality has a different corresponding camera angle with respect to the patient; calculating calibration data for the camera for each of the reflection images; forming a sparse point cloud corresponding to the reflection images according to a multi-view geometry; automatically registering the sparse point cloud to the dense point cloud; mapping texture data from the reflection images to the dense point cloud; and displaying the texture-mapped volume image.

While the invention has been illustrated with respect to one or more implementations, alterations and/or modifications can be made to the illustrated examples without departing from the spirit and scope of the appended claims. In addition, while a particular feature of the invention can have been disclosed with respect to one of several implementations, such feature can be combined with one or more other features of the other implementations as can be desired and advantageous for any given or particular function. The term “at least one of” is used to mean one or more of the listed items can be selected. The term “about” indicates that the value listed can be somewhat altered, as long as the alteration does not result in nonconformance of the process or structure to the illustrated embodiment. Finally, “exemplary” indicates the description is used as an example, rather than implying that it is an ideal. Other embodiments of the invention will be apparent to those skilled in the art from consideration of the specification and practice of the invention disclosed herein. The presently disclosed embodiments are therefore considered in all respects to be illustrative and not restrictive. The scope of the invention is indicated by the appended claims, and all changes that come within the meaning and range of equivalents thereof are intended to be embraced therein.

1. A method for forming a 3-D facial model, the method executed at least in part on a computer and comprising:
obtaining a reconstructed radiographic image volume of at least a portion of the head of a patient;
extracting a soft tissue surface of the patient's face from the reconstructed radiographic image volume and forming a dense point cloud corresponding to the extracted soft tissue surface;
acquiring a plurality of reflection images of the face using a camera, wherein each reflection image has a different corresponding camera angle with respect to the patient and calculating calibration data for the camera for one or more of the reflection images;
forming a sparse point cloud corresponding to the reflection images by processing the reflection images using multi-view geometry and the calculated calibration data;
registering the sparse point cloud to the dense point cloud and calculating a transformation between reflection image texture data and the dense point cloud;
applying the calculated transformation for mapping texture data from the plurality of reflection images to the dense point cloud to form a texture-mapped volume image; and
displaying the texture-mapped volume image.

2. The method of claim 1 wherein the radiographic image volume is from a computed tomography cone-beam imaging apparatus, and wherein the reflection images are acquired using a digital camera.

3. The method of claim 1 wherein the calibration data for the camera comprises imaging characteristics that correlate three-dimensional spatial coordinates with two-dimensional camera pixel coordinates.

4. The method of claim 1 further comprising:
transmitting or storing the texture-mapped volume image; and
modifying the transparency of the mapped texture data, wherein forming the sparse point cloud further comprises applying a structure from motion algorithm.

5. The method of claim 1 wherein automatically registering the sparse point cloud is automatically registered to the dense point cloud.

6. A method for forming a 3-D facial model, the method executed at least in part on a computer and comprising:
forming a first point cloud of the patient's face from a reconstructed radiographic volume image of the patient;
forming a second point cloud of the patient's face from a plurality of reflectance images of the patient, using a structure-from-motion logic sequence;
registering the first point cloud to the second point cloud; and
mapping image texture content from one or more of the plurality of reflectance images according to the point-cloud registration and displaying the mapping of image texture content.

7. The method of claim 6 wherein forming the second point cloud further comprises obtaining camera calibration data.

8. The method of claim 6 further comprising transmitting or storing the texture-mapped volume image, wherein the radiographic image volume is from a computed tomography cone-beam imaging apparatus.

9. An apparatus for generating a 3-D facial model of a patient, the apparatus comprising:
a computed tomography imaging apparatus comprising;
a transport apparatus that is energizable to rotate a radiation source and an imaging detector about the patient;
a control logic processor in signal communication with the transport apparatus and responsive to stored instructions for:
(i) rotating the radiation source and detector about the patient and acquiring a plurality of radiographic images;
(ii) forming a volume image and a dense point cloud according to the acquired plurality of radiographic images;
(iii) accepting a plurality of reflectance images that are acquired from a camera that is moved about the patient;
(iv) generating a sparse point cloud that is registered to the dense point cloud according to the plurality of reflectance images;
(v) mapping texture content to the dense point cloud from the plurality of reflectance images to form texture-mapped volume images;
and
a display that is in signal communication with the control logic processor and that displays one or more of the texture-mapped volume images.

10. The apparatus of claim 9 wherein the computed tomography imaging apparatus is a cone-beam computed tomography imaging apparatus, and wherein the camera is coupled to the transport apparatus.
